(19) United States
(12) Patent Application Publication
(10) Pub. No.: US 2020/0075163 A1
(43) Pub. Date: Mar. 5, 2020

(54) DIAGNOSTIC DECISION SUPPORT FOR PATIENT MANAGEMENT

(71) Applicant: International Business Machines Corporation, Armonk, NY (US)

(72) Inventors: Ella Barkan, Haifa (IL); Murray A. Reicher, Rancho Santa Fe, CA (US); Eugeniusz Walach, Haifa (IL)

(21) Appl. No.: 16/120,658

(22) Filed: Sep. 4, 2018

(51) Int. Cl. G16H 50/20; G06Q 30/02; G16H 10/60

(52) U.S. Cl. CPC G16H 50/20; G16H 10/60; G06Q 30/0283

(57) ABSTRACT

A diagnostic manager receives a health history of a patient and generates a plurality of probabilities corresponding to a plurality of medical conditions based on the health history of the patient. The diagnostic manager generates a first list of potential diagnostic measures and calculates respective values for respective diagnostic measures and respective costs for respective diagnostic measures in the first list of potential diagnostic measures. The diagnostic manager calculates respective scores for respective diagnostic measures in the first list of potential diagnostic measures and outputs a recommended diagnostic measure.

### BACKGROUND

The present disclosure relates to patient management, and, more specifically, to diagnostic decision-making support for healthcare practitioners.

Clinical decision support systems (CDSS) are information technology (IT) medical tools that can provide physicians with standardized, automated, and/or evidence-based decision-making support. CDSS includes technologies such as, but not limited to, computer-aided detection (e.g., CADe), computer-aided diagnosis (e.g., CADx), computer-aided diagnostic quantification (e.g., CADq), and/or computer-aided simple triage (e.g., CAST). Collectively, these IT medical tools can support consistent, efficient, and safe medical care.

### SUMMARY

Aspects of the present disclosure are directed toward a method comprising receiving, by a clinical decision support system (CDSS) executed on a computer, a health history of a patient, the health history comprising a plurality of known diagnostic measures. The method can further comprise generating, by the CDSS, a first number of a first plurality of probabilities corresponding to a plurality of medical conditions based on the health history of the patient. The method can further comprise generating, by the CDSS, a first list of potential diagnostic measures by removing the plurality of known diagnostic measures from a second list of all diagnostic measures. The method can further comprise calculating, by the CDSS, respective values for respective diagnostic measures in the first list of potential diagnostic measures. The method can further comprise calculating, by the CDSS, respective costs for respective diagnostic measures in the first list of potential diagnostic measures. The method can further comprise calculating, by the CDSS, respective scores for respective diagnostic measures in the first list potential of diagnostic measures, where respective scores are based on respective values and respective costs. The method can further comprise outputting, by the CDSS and to a display of the computer, a recommended diagnostic measure having a highest score.

Other aspects of the present disclosure are directed toward a system and a computer program product having similar characteristics as those described above. The present Summary is not intended to illustrate each aspect of, every implementation of, and/or every embodiment of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 illustrates a flowchart of an example method for outputting a recommended diagnostic measure, in accordance with embodiments of the present disclosure.

FIG. 2 illustrates a flowchart of an example method for outputting a subsequent recommended diagnostic measure, in accordance with embodiments of the present disclosure.

FIG. 3 illustrates a flowchart of an example method for determining a value of a diagnostic measure, in accordance with embodiments of the present disclosure.

FIG. 4 illustrates a flowchart of an example method for determining a cost of a diagnostic measure, in accordance with embodiments of the present disclosure.

FIG. 5 illustrates a block diagram of an example diagnostic manager, in accordance with embodiments of the present disclosure.

While the present disclosure is amenable to various modifications and alternative forms, specifics thereof have been shown by way of example in the drawings and will be described in detail. It should be understood, however, that the intention is not to limit the present disclosure to the particular embodiments described. On the contrary, the intention is to cover all modifications, equivalents, and alternatives falling within the spirit and scope of the present disclosure.

### DETAILED DESCRIPTION

Aspects of the present disclosure are directed toward patient management, and, more specifically, to diagnostic decision-making support for healthcare practitioners. While not limited to such applications, aspects of the present disclosure may be better appreciated in light of the aforementioned applications.

Aspects of the present disclosure relate to a clinical decision support system (CDSS) for efficiently reaching a medical diagnosis. Advantageously, aspects of the present disclosure provide automated, evidence-based recommendations for diagnostic measures (e.g., questions, tests, etc.) that can result in a medical diagnosis with a relatively high certainty and a relatively low cost. Thus, aspects of the present disclosure improve the technology of medical diagnostics.

Aspects of the present disclosure can be particularly useful in the field of differential diagnosis. Differential diagnosis can refer to making a medical diagnosis by eliminating possible diseases until a single disease remains. Thus, aspects of the present disclosure are particularly useful in reaching a medical diagnosis by differential diagnosis where a physician may be unable to identify beneficial diagnostic measures from hundreds or thousands of possible diagnostic measures to efficiently reach a differential diagnosis.

Aspects of the present disclosure realize improvements in medical diagnostics. For example, aspects of the present disclosure reduce time in reaching an accurate medical diagnosis, reduce costs associated with making an accurate medical diagnosis, and/or reduce patient side-effects associated with making an accurate medical diagnosis.

Aspects of the present disclosure realize the aforementioned advantages by quantifying a value of the possible diagnostic measures and quantifying a cost of the possible diagnostic measures. The value can relate to, among other things, a magnitude of a difference in the differential diagnosis given an answer to a diagnostic measure (e.g., an increase in the probability of a particular disease given a first answer, a decrease in the number of possible diseases given a second answer, etc.). The cost can relate to, among other things, availabilities, financial costs, and/or side-effects associated with the diagnostic measure. Thus, aspects of the present disclosure can determine a beneficial recommended diagnostic measure by quantifying values and costs for various diagnostic measures.

Furthermore, aspects of the present disclosure realize improvements relative to other CDSSs. As a first example, aspects of the present disclosure account for both values and costs of diagnostic measures, thereby balancing the need for accurate information with financial, availability, and/or safety constraints. As a second example, aspects of the present disclosure can calculate values and costs for a series of diagnostic measures. Thus, aspects of the present disclosure can identify a most useful series of diagnostic measures. For example, a first diagnostic measure could be most beneficial to the differential diagnosis when considered individually, whereas a set of diagnostic measures not including the first diagnostic measure could be most beneficial to the differential diagnosis when considered collectively. As a third example, aspects of the present disclosure utilize independent diagnostic measures, thereby reducing the total number of diagnostic measures and reducing memory usage, increasing processing speed, and/or realizing other computational enhancements. As a fourth example, aspects of the present disclosure reduce memory usage and increase processing efficiency by storing the differential diagnosis as a vector where each element of the vector corresponds to a probability of a respective medical condition. Vectors are well-suited to the field of automated differential diagnosis because they have low memory usage, good locality of reference, and good data cache utilization. Furthermore, vectors provide numerous strategies for quantifying a value of a diagnostic measure. For example, a component of the vector can be used as a value (e.g., a largest element), a difference between two components can be used as a value (e.g., a largest difference between any two corresponding elements), a magnitude of a difference vector can be used as a value (e.g., where the difference vector includes each difference for each pair of corresponding elements in two vectors, and where the magnitude comprises a square root of a sum of each squared element in the difference vector), and so on. Thus, aspects of the present disclosure utilizing vectors realize improvements in processing speed, memory efficiency, and quantification flexibility.

The aforementioned advantages are example advantages, and embodiments exist that can contain all, some, or none of the aforementioned advantages while remaining within the spirit and scope of the present disclosure.

Before discussing the figures, it may be helpful to discuss some of the terms used in the present disclosure. As used herein, the term “medical diagnosis” can refer to which disease, condition, illness, sickness, syndrome or other medical condition explains a patient's symptoms. A medical diagnosis can be determined based on test results, questions, medical histories, and other relevant information about a patient. Identifying accurate medical diagnoses efficiently (e.g., in a short amount of time and/or with limited costs) improves patient health (e.g., diseases identified early can be treated with higher success rates) and conserves resources (e.g., excessive diagnostic testing can be expensive, time-consuming, and/or unsafe).

As used herein, the term “diagnostic measure” can refer to any diagnostic procedure, test, and/or question useful for procuring relevant information to diagnosing a medical condition. Thus, a diagnostic measure can include images (e.g., photographs, x-rays, computed tomography (CT) scans, mammograms, magnetic resonance imaging (MRI) scans, etc.), tests (e.g., blood tests, cholesterol tests, blood pressure tests, pulse rate tests, biopsies, etc.), and/or questions (e.g., “does a parent or grandparent have diabetes?”, “how many alcoholic drinks did you consume in the last month?”, etc.).

As used herein, the term “health history” can refer to historical information from a patient including, for example, medical information, demographic information, personal information, and/or social information.

Medical information can include, but is not limited to, medical diagnoses, medical prescriptions (e.g., medication type, medication dosage, medication duration, etc.), medical procedures, medical diagnostics (e.g., blood pressure, pulse rate, cholesterol levels, blood type, mammograms, CT scans, x-ray scans, MRI scans, biopsies, etc.), allergies, genetic information, and other medical information.

Demographic information can include, but is not limited to, race, ethnicity, gender, age, education, profession, occupation, income level, marital status, and so on.

Personal information can include, but is not limited to, height, weight, body mass index (BMI), hair color, eye color, birth date, social security number, address, eating habits, sleeping habits, exercising habits, and so on.

Social information can include, but is not limited to, family dynamics, friendships, relationships, community information, and other information.

Diagnostic measures can be associated with health history information. For example, a blood pressure reading for a patient contained in a health history of the patient can be associated with one or more diagnostic measures (e.g., a first diagnostic measure asking the patient “what is your typical blood pressure?” and a second diagnostic measure instructing a physician “take the patient's blood pressure at the brachial artery using the auscultatory method or the oscillometric method”). Diagnostic measures can be correlated with health history information inputs in order to populate the diagnostic measures with information already stored in the health history information (e.g., in order to exclude those diagnostic measures for which the information already exists).

In some embodiments, diagnostic measures are associated with predetermined answers. For example, questions can be associated with three possible answers such as “Yes”, “No”, and “I don't know”. Likewise, tests involving numbers can be associated with ranges or classifications. For example, a blood pressure test can ask for input of systolic and diastolic blood pressure numbers, and automatically classify those numbers as normal, pre-hypertension, or hypertension. Alternatively, a diagnostic measure related to blood pressure can ask, “Is your systolic blood pressure above 140 mm Hg?” Thus, embodiments of the present disclosure can limit potential answers of some diagnostic measures in order to accurately model the effect of each answer on a probability of one or more diseases when performing differential diagnosis.

As used herein, the term “medical condition” can relate to an abnormal physical, mental, and/or emotional condition that impairs, negatively affects, or otherwise compromises normal, regular, or expected functioning of a patient. Medical conditions can include, for example, diseases, disorders, illnesses, syndromes, and/or sicknesses. Medical conditions can be associated with acute (e.g., short-term), chronic (e.g., long-term), congenital (e.g., present at birth), genetic (e.g., caused by a genetic mutation), hereditary (e.g., caused by a hereditary genetic mutation), iatrogenic (e.g., caused by medical intervention), idiopathic (e.g., unknown cause), and/or other characteristics.

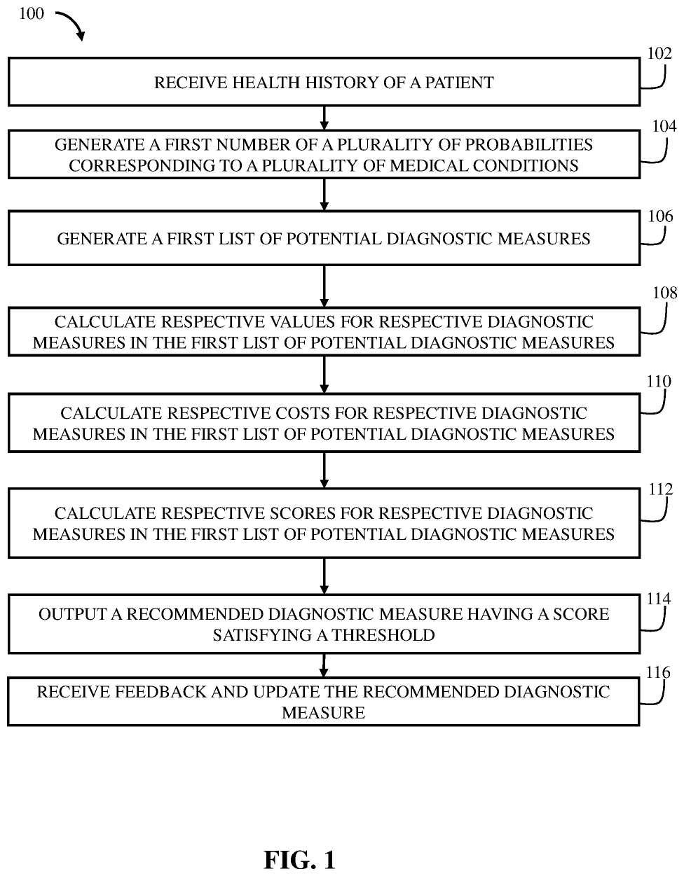

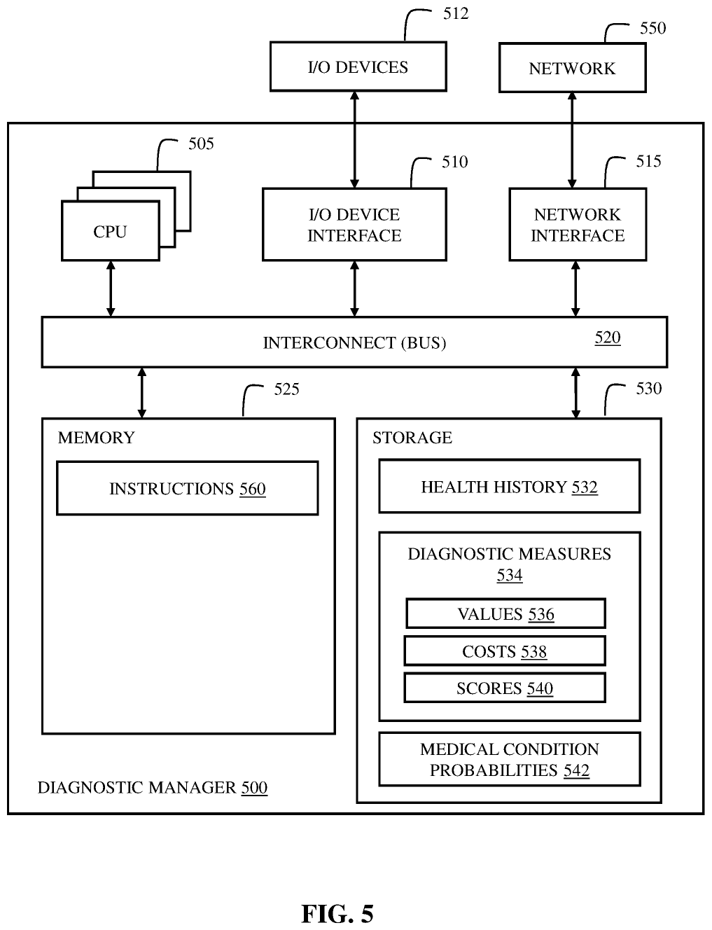

FIG. 1 illustrates a flowchart of an example method 100 for recommending diagnostic measures, in accordance with some embodiments of the present disclosure. Although the method 100 is discussed as being performed by a diagnostic manager (e.g., diagnostic manager 500 of FIG. 5), the method 100 can also be performed by alternative configurations of hardware and/or software.

In operation 102, the diagnostic manager can receive a health history of a patient. The health history can be received from a database storing patient information (e.g., an electronic medical record (EMR), an electronic health record (EHR), etc.), from manual input (e.g., responsive to a manual or digital questionnaire), from data mining (e.g., retrieving information from a social media platform, etc.), by a combination of the aforementioned techniques, or by a different technique.

In operation 104, the diagnostic manager can generate a first number of a plurality of probabilities corresponding to a plurality of medical conditions. The first number can refer to a first quantity of respective probabilities for respective medical conditions. The first number can include respective probabilities above a first threshold (e.g., the first threshold comprising a minimum probability of, for example, 1%, 5%, 10%, 25%, 50%, or a different probability). In some embodiments, the first threshold is user-defined. In some embodiments, the first threshold is dynamic (e.g., only the highest three probabilities are shown). In some embodiments, the probabilities are shown as a percentage with a predefined precision (e.g., 32.5%). In other embodiments, the probabilities are shown as a decimal number (e.g., 0.325). In some embodiments, probabilities below a threshold are shown as zero (e.g., a probability of 0.0001 or 0.01% can be considered a probability of 0). Such changes in precision can be defined by rounding commands on numerical data having a floating-point number data structure (e.g., float). In other embodiments, the precision can be defined by the type of numerical data structure. For example, an integer numerical data structure (e.g., int) automatically rounds down (e.g., 2.9 becomes 2). In such cases, any probability below 1% can be considered 0.

In some embodiments, the plurality of probabilities are output as a vector, where respective positions (e.g., elements) in the vector correspond to respective medical conditions. Vectors can be advantageous because vectors have low memory usage, good locality of reference, good data cache utilization, and vectors allow random access. For these reasons, embodiments utilizing vectors to store the first number of the plurality of probabilities can realize improved memory efficiency and/or processing speed relative to alternative methods (e.g., rows of a database).

Operation 104 can utilize algorithms, models, machine learning, and/or other techniques to determine the plurality of probabilities. In some embodiments, operation 104 includes applying weights to separate types of health history information indicating the relevance of the type of health history information in determining a probability that the patient has a certain medical condition. In some embodiments, operation 104 includes generating confidence intervals associated with each probability and indicating a range within which the true probability resides at a given certainty (e.g., 95% certainty, 99% certainty, etc.). In some embodiments, operation 104 can utilize one or more of decision tree learning, association rule learning, artificial neural networks, deep learning, inductive logic programming, clustering, support vector machines (SVMs), Bayesian networks, reinforcement learning, representation learning, genetic algorithms, rule-based machine learning, or a different machine learning approach to generate the plurality of probabilities.

In operation 106, the diagnostic manager can generate a first list of potential diagnostic measures. The first list of potential diagnostic measures can be generated by excluding each diagnostic measure that is already accounted for based on the health history of the patient. For example, diagnostic measures related to “blood pressure” (e.g., a question asking the patient about their blood pressure, an instruction for a physician to measure a patient's blood pressure, etc.) may be excluded from the list of potential diagnostic measures if a recent blood pressure reading is included in the health history of the patient. In some embodiments, decisions on whether to exclude diagnostic measures can be based on a combination of the presence of the diagnostic measure in the health history and an amount of time that has elapsed since the diagnostic measure was taken. For example, if a mammogram exists in a patient's health history, but the mammogram was taken five years ago, then operation 106 need not exclude diagnostic measures related to mammograms since the duration of time that has passed since the mammogram was performed is above a threshold (e.g., 1 year, 3 years, etc.).

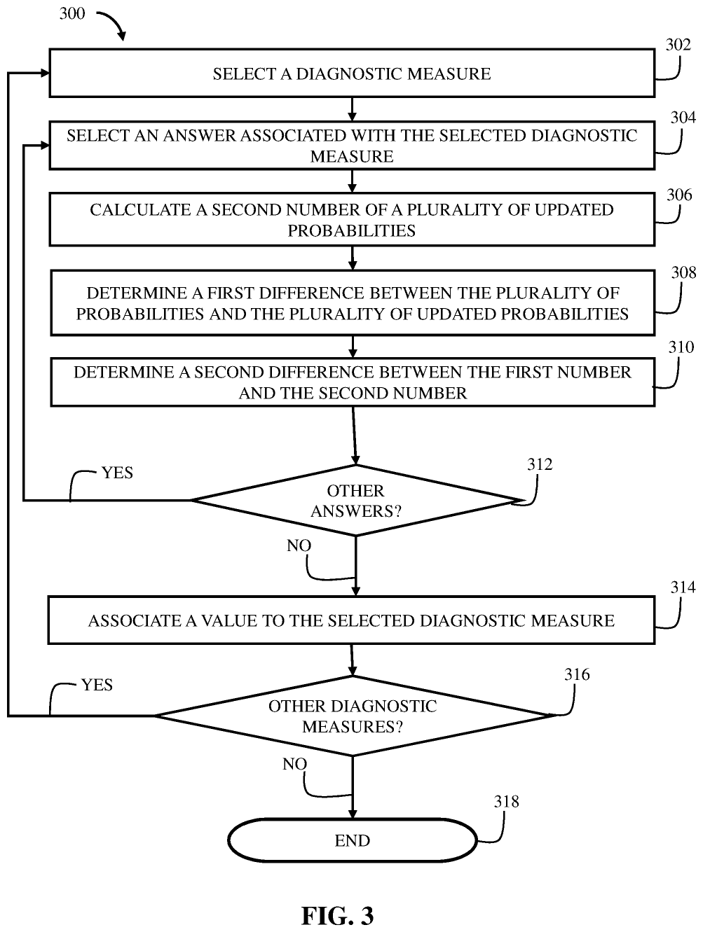

In operation 108, the diagnostic manager can calculate respective values for respective diagnostic measures in the first list of potential diagnostic measures. Values can quantify the anticipated benefit in accurately diagnosing a patient's medical condition. For example, values can quantify an improvement in a probability (e.g., an increase in probability), an improvement in confidence (e.g., a decreased size of a confidence interval), and/or a reduction in the number of possible medical conditions (e.g., reducing possible medical conditions from 38 to 12). Operation 108 is discussed in more detail hereinafter with respect to FIG. 3.

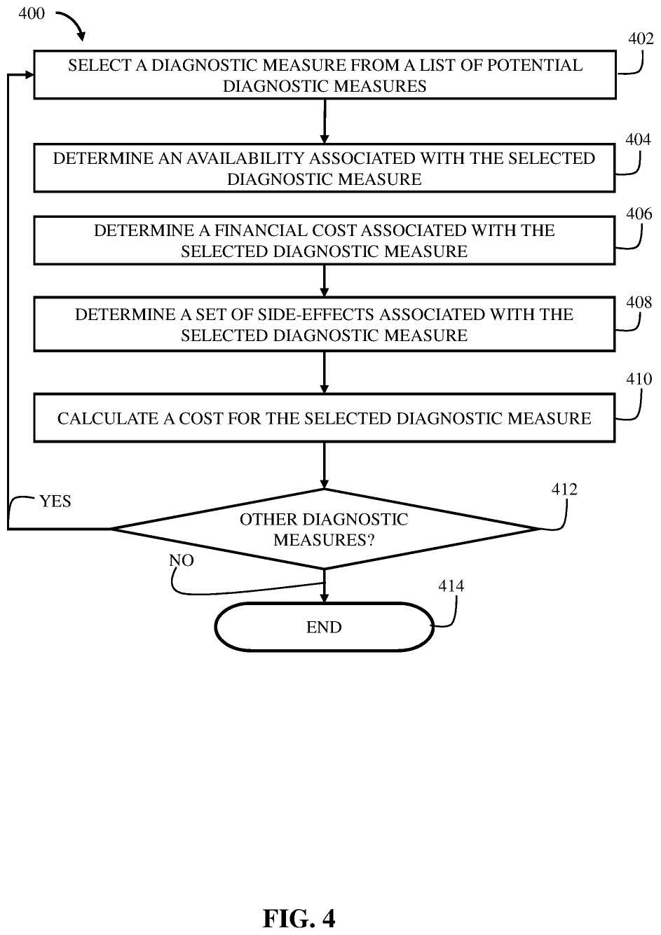

In operation 110, the diagnostic manager can calculate respective costs for respective diagnostic measures in the first list of potential diagnostic measures. Costs can quantify the availability, financial burden, and/or side-effects associated with a diagnostic measure. For example, asking a question may have a low cost compared to performing an invasive diagnostic procedure. Operation 110 is discussed in more detail hereinafter with respect to FIG. 4.

In operation 112, the diagnostic manager can calculate respective scores for respective diagnostic measures in the first list of potential diagnostic measures. In some embodiments, the scores comprise a ratio of a value (e.g., from operation 108) and a cost (e.g., from operation 110) for a same diagnostic measure. For example, if a diagnostic measure in the form of question “does a parent or grandparent have glaucoma” has a value of 3 and a cost of 1, then a score comprising the ratio of value/cost for the aforementioned example is 3. As an alternative example, if a diagnostic measure “perform ultrasound of midsection” has a value of 9 and a cost of 4, then the score comprising the ratio of value/cost for the aforementioned example is 2.25. Although value/cost ratios were previously described, some embodiments can utilize a cost/value ratio, or combine the value and cost in a different algorithm that may weight, normalize, and/or otherwise combine the value and the cost to generate a score.

In operation 114, the diagnostic manager can output a recommended diagnostic measure having a score satisfying a threshold. In embodiments where the score comprises a value/cost ratio, operation 114 can output a recommended diagnostic measure having a score above the threshold. In embodiments where the score comprises a cost/value ratio, operation 114 can output a recommended diagnostic measure having a score below the threshold. In some embodiments, operation 114 outputs a best (e.g., highest, lowest, etc.) score or a predefined number (e.g., 3, 5, etc.) of the best scores.

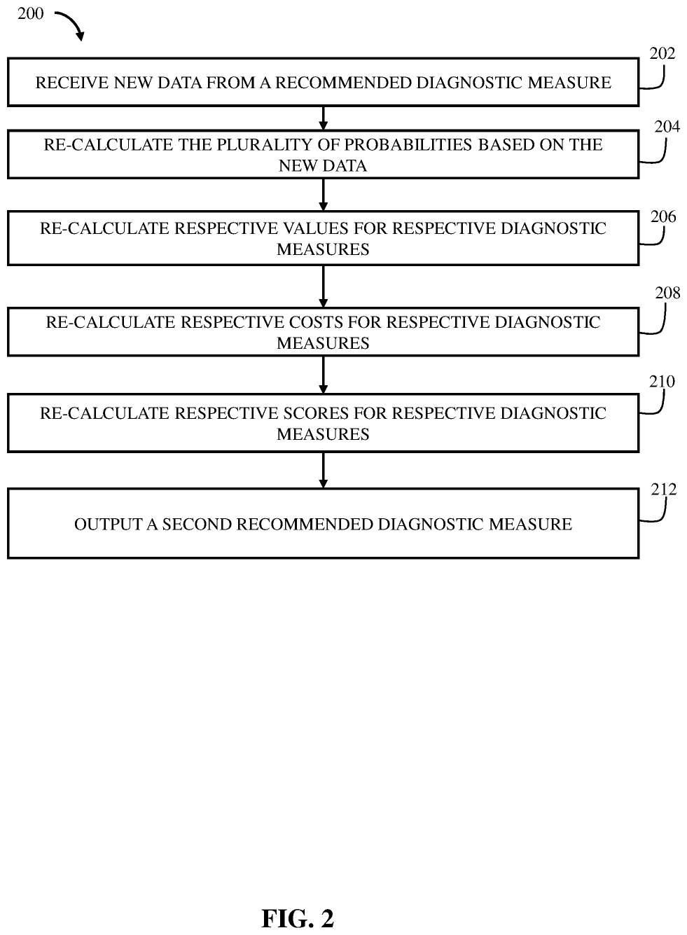

Outputting a recommended diagnostic measure can include, but is not limited to, presenting, on a user display, the recommended diagnostic measure and optionally including any additional information (e.g., probabilities of various medical conditions, values, costs, scores, etc.). In some embodiments, outputting a recommended diagnostic measure includes transmitting, from the diagnostic manager and via a wired or wireless communication channel, the recommended diagnostic measure to a computer, server, laptop, and/or user device. In some embodiments, outputting a recommended diagnostic measure includes storing the recommended diagnostic measure in a tangible computer readable storage medium in a file format that can be retrieved, opened, and/or viewed by a computer, laptop, server, and/or user device communicatively coupled to the tangible computer readable storage medium and functioning with an operating system (OS) that is similar or dissimilar to the OS of the diagnostic manager. In embodiments that generate a file as part of the recommended diagnostic measure, the file can include values, costs, scores, patient information, probabilities of various medical conditions, dates, times, and/or other information. In some embodiments, the recommended diagnostic measure includes a series of recommended diagnostic measures (e.g., discussed in more detail hereinafter with respect to FIG. 2).

In operation 116, the diagnostic manager can receive feedback. Feedback can include information related to the recommended diagnostic measure output in operation 114 and/or feedback from a physician interacting with the diagnostic manager. In either case, the feedback can be ingested and the recommended diagnostic measure can be updated accordingly (if it is updated at all). For example, the feedback can include an answer to a question where the recommended diagnostic measure was the question. In this example, the method 100 can again proceed through operations 104-114 to output a second recommended diagnostic measure based on the answer to the question. As another example, the feedback can include a physician creating a setting such that only the five medical conditions with the highest probabilities are shown rather than all medical conditions with a probability exceeding a threshold. Thus, feedback can include any one, or any combination of, test results, answers to questions, physician preferences, and so on.

FIG. 1 is intended to illustrate the representative operations of an example method for recommending a diagnostic measure. However, in some embodiments, more, fewer, and/or different operations than the operations shown in FIG. 1 are performed while remaining within the spirit and scope of the present disclosure.

FIG. 2 illustrates a flowchart of an example method 200 for outputting a second recommended diagnostic measure, in accordance with embodiments of the present disclosure. In some embodiments, the method 200 occurs after operation 116 of FIG. 1. Although the method 200 is discussed as being performed by a diagnostic manager (e.g., diagnostic manager 500 of FIG. 5), the method 200 can also be performed by alternative configurations of hardware and/or software.

In operation 202, the diagnostic manager can receive new data from a previously recommended diagnostic measure. For example, if the diagnostic manager previously recommended a CT scan of a patient's head, the new data received in operation 202 can be the completed CT scan. As another example, if the diagnostic manager previously recommended asking the question “have you had a fever in the last seven days?”, the new data can be the patient's answer to the question (e.g., “yes”, “no”, “I don't know”, etc.).

In operation 204, the diagnostic manager can re-calculate the plurality of probabilities based on the health history of the patient and the new data received in operation 202. Operation 204 can utilize the same or similar algorithms, models, and/or machine learning techniques as previously discussed with respect to operation 104 of FIG. 1.

In operation 206, the diagnostic manager can re-calculate the respective values for respective diagnostic measures from the first list of potential diagnostic measures (e.g., from operation 106 of FIG. 1), where the first list of potential diagnostic measures has the previously recommended diagnostic measure removed (e.g., if the previously recommended diagnostic measure was a CT scan, and a CT scan was received in operation 202, then the updated first list of potential diagnostic measures can exclude the CT scan diagnostic measure). Operation 206 can calculate values in a same or similar manner as previously discussed with respect to operation 108 of FIG. 1 and/or as further discussed hereinafter with respect to FIG. 3.

In operation 208, the diagnostic manager can re-calculate respective costs for respective diagnostic measures in the first list of potential diagnostic measures with the previously recommended diagnostic measure excluded. Operation 208 can calculate costs in a same or similar manner as previously discussed with respect to operation 110 of FIG. 1 and/or as further discussed hereinafter with respect to FIG. 4.

In operation 210, the diagnostic manager can re-calculate respective scores for respective diagnostic measures included in the first list of potential diagnostic measures with the previously recommended diagnostic measure excluded. Operation 210 can calculate scores in a same or similar manner as previously discussed with respect to operation 112 of FIG. 1 (e.g., a value/cost ratio, a cost/value ratio, or a different algorithm accounting for both the cost and the value for respective diagnostic measures).

In operation 212, the diagnostic manager can output a second recommended diagnostic measure. The second recommended diagnostic measure can have a best score, or a score satisfying a threshold (e.g., above the threshold, below the threshold, within a threshold range, equaling the threshold, etc.). The second recommended diagnostic measure can be output in a same or similar manner as operation 114 of FIG. 1.

Although FIG. 2 generally describes an example method for recommending a subsequent diagnostic measure, FIG. 2 can also outline the operations for outputting a recommended series of diagnostic measures (e.g., two or more sequential diagnostic measures). In such embodiments, operation 202 can use possible answers from a first recommended diagnostic measure (e.g., if the first recommended diagnostic measure is a question, then operation 202 can input “yes”, “no” and “I don't know” as the possible answers to the question). The method 200 can then proceed through operations 204-210 for each possible answer to the first recommended diagnostic measure. This process can be repeated for any number of first recommended diagnostic measures (e.g., the top three recommended diagnostic measures from FIG. 1). In these embodiments, operation 212 can include combining two or more diagnostic measures into a recommended series of diagnostic measures based on cumulative values and costs of the series of recommended diagnostic measures. Although a series of two diagnostic measures is described above, the series can be any number of diagnostic measures.

FIG. 2 is intended to represent the major components of an example method for outputting a second recommended diagnostic measure, in accordance with embodiments of the present disclosure. However, in some embodiments, more, fewer, and/or different operations than the operations shown in FIG. 2 are performed while remaining within the spirit and scope of the present disclosure.

FIG. 3 illustrates a flowchart of an example method 300 for calculating values, in accordance with embodiments of the present disclosure. In some embodiments, the method 300 is a sub-method of operation 108 of FIG. 1 and/or operation 206 of FIG. 2. Although the method 300 is discussed as being performed by a diagnostic manager (e.g., diagnostic manager 500 of FIG. 5), the method 300 can also be performed by alternative configurations of hardware and/or software.

In operation 302, the diagnostic manager can select a diagnostic measure from the first list of potential diagnostic measures.

In operation 304, the diagnostic manager can select an answer associated with the selected diagnostic measure. For example, if the selected diagnostic measure comprises the question “have you had a fever in the last seven days”, operation 304 can select one of three possible answers: “yes”, “no”, and “I don't know”. As another example, if the diagnostic measure comprises “perform a CT scan to confirm presence of a tumor in the lung”, operation 304 can select one of three possible answers: “yes, there is a tumor on the lung according to the CT scan”, “no tumor is visible on the lung according to the CT scan”, or “cannot determine whether a tumor is on the lung according to the CT scan”.

In operation 306, the diagnostic manager can calculate a second number of a second plurality of updated probabilities (e.g., respective probabilities of respective diseases given the selected answer from operation 304). Operation 306 can utilize similar algorithms, models, and/or machine learning techniques to estimate the probabilities as used in operation 104 of FIG. 1.

In operation 308, the diagnostic manager can determine a first difference between the first plurality of probabilities (e.g., from operation 104 of FIG. 1) and the second plurality of updated probabilities (e.g., from operation 306 of FIG. 3). In some embodiments, the first difference comprises a difference vector storing a respective difference for each corresponding pair of probabilities between the first plurality of probabilities and the second plurality of updated probabilities. In some embodiments, the first difference comprises a largest difference of any corresponding pair of probabilities between the first plurality of probabilities and the second plurality of updated probabilities. In other embodiments, the first difference can comprise an average difference accounting for all corresponding pairs of probabilities for one or all possible answers to a selected diagnostic measure. In other embodiments, the difference can be a magnitude of the difference vector for a selected answer, or an average magnitude of the difference vector for all possible answers to a selected diagnostic measure. In embodiments including a magnitude of the difference vector, the magnitude can be calculated by determining a square root of a sum of squared elements of the difference vector. Thus, operation 308 can determine a value by quantifying changes in probabilities of certain medical conditions based on selected answers to selected diagnostic measures.

In operation 310, the diagnostic manager can determine a second difference between the first number (e.g., the original quantity of possible medical conditions having a probability exceeding the threshold) and the second number (e.g., the updated quantity of possible medical conditions having a probability exceeding the threshold). In some embodiments, operation 310 includes determining a first size of the first vector, where the first size comprises a first number of non-zero elements in the first vector. In such embodiments, operation 310 further includes determining a second size of the second vector, where the second size corresponds to a second number of non-zero elements in the second vector. In such embodiments, the second difference determined in operation 310 is the difference in the first number of non-zero elements in the first vector (e.g., the first size) relative to the second number of non-zero elements in the second vector (e.g., the second size). Thus, operation 310 quantifies how many possible medical conditions can be ruled out as a result of a particular answer to a particular diagnostic measure.

In some embodiments, the value is based on both differences in probabilities of medical conditions and differences in quantities of medical conditions, and, in such embodiments, operations 308 and 310 both occur. In other embodiments, however, operations 308 and 310 need not both occur. For example, in some embodiments, the value is based on differences in probabilities (e.g., according to operation 308), whereas, in other embodiments, the value is based on differences in quantities of possible medical conditions (e.g., according to operation 310).

In operation 312, the diagnostic manager can determine if there are additional answers for the selected diagnostic measure. If there are additional answers, the method 300 returns to operation 304 and selects a new answer for the selected diagnostic measure. If there are no more possible answers to the selected diagnostic measure (e.g., all possible answers have been previously selected), then the method 300 can proceed to operation 314.

Operation 314 can associate a value to a selected diagnostic measure. In some embodiments, the value is based on the first difference from operation 308 and comprises the largest difference of any pair of corresponding probabilities for any medical condition based on any single answer for any diagnostic measure. In other embodiments, the value comprises a largest magnitude of the difference vector for a selected answer to a given diagnostic measure. In other embodiments, the value comprises an average magnitude of the difference vector accounting for each possible answer of a given diagnostic measure.

In some embodiments, the value is based on the second difference from operation 310 and comprises the largest single change in possible medical conditions for any single answer for a selected diagnostic measure. In other embodiments, the value comprises the largest average change in possible medical conditions accounting for every answer for a selected diagnostic measure. In some embodiments, the value is based on both the first difference and the second difference.

In operation 316, the diagnostic manager can determine if there are other diagnostic measures. If so, the method 300 returns to operation 302 and selects another diagnostic measure. If not, the method 300 proceeds to operation 318 and ends.

The method 300 is intended to represent the major operations of an example method for associating a value to a diagnostic measure, in accordance with some embodiments of the present disclosure. However, in some embodiments, more, fewer, and/or different operations than the operations shown in FIG. 3 are performed while remaining within the spirit and scope of the present disclosure.

FIG. 4 illustrates a flowchart of an example method 400 for associating costs to respective diagnostic measures, in accordance with some embodiments of the present disclosure. In some embodiments, the method 400 is a sub-method of operation 110 of FIG. 1 and/or operation 208 of FIG. 2. Although the method 400 is discussed as being performed by a diagnostic manager (e.g., diagnostic manager 500 of FIG. 5), the method 400 can also be performed by alternative configurations of hardware and/or software.

In operation 402, the diagnostic manager can select a diagnostic measure from a list of potential diagnostic measures.

In operation 404, the diagnostic manager can determine an availability associated with the selected diagnostic measure. Availabilities can be determined by, for example, determining whether or not the medical facility associated with the patient has the capability to perform the diagnostic measure. For example, if the diagnostic measure is a question, then the diagnostic measure is associated with a high availability. In contrast, if the diagnostic measure is a nerve conduction velocity (NCV) test, for example, the patient may not be in a location having the proper equipment to conduct such a test, therefore, the availability can be considered low.

In some embodiments, operation 404 utilizes a threshold to determine availability such as, for example, determining if the selected diagnostic measure can be performed within one day from the present time (or a different interval, such as one week). In other embodiments, operation 404 utilizes a threshold distance to determine availability such as, for example, determining if the selected diagnostic measure can be performed at a medical facility within 50 miles of the patient's address.

In operation 406, the diagnostic manager can determine a financial cost associated with the selected diagnostic measure. Financial costs can be total financial costs, or financial costs realized by the patient after accounting for health insurance benefits. Operation 406 can use patient health history information to determine health insurance coverage and calculate patient expenses accordingly.

In operation 408, the diagnostic manager can determine a set of side-effects associated with the selected diagnostic measure. Side-effects can include, for example, long-term physical side effects (e.g., radiation from a CT scan or X-ray image), short-term physical side effects (e.g., discomfort as a result of an invasive medical procedure), inconvenience side-effects (e.g., fasting for 12 hours prior to a blood test), and/or other side-effects. In some embodiments, operation 408 includes requesting and receiving feedback from the patient such as, for example, “are you comfortable having this diagnostic procedure?” Such a question can be beneficial to identify other non-obvious side-effects. For example, a patient with claustrophobia should not undergo a CT scan unless necessary.

In operation 410, the diagnostic manager can calculate a cost for the selected diagnostic measure based on one or more of the availability of the diagnostic measure, the financial cost of the diagnostic measure, and/or the set of side-effects associated with the diagnostic measure. Operation 410 can utilize any number of algorithms for adding, weighting, normalizing, or otherwise combining the aforementioned factors.

In operation 412, the diagnostic manager can determine if there are other diagnostic measures to be evaluated. If so, the method 400 can return to operation 402 and select a new diagnostic measure. If not, the method 400 can proceed to operation 414 and end.

FIG. 4 is intended to illustrate the major operations of an example method for determining a cost associated with a diagnostic measure, in accordance with embodiments of the present disclosure. However, in some embodiments, more, fewer, and/or different operations than the operations shown in FIG. 4 are performed while remaining within the spirit and scope of the present disclosure.

FIG. 5 illustrates a block diagram of an example diagnostic manager 500 in accordance with some embodiments of the present disclosure. In various embodiments, diagnostic manager 500 can perform the methods 100, 200, 300, and/or 400 as described in FIGS. 1-4. In some embodiments, diagnostic manager 500 provides instructions for any of the methods 100, 200, 300, and/or 400 of FIGS. 1-4 to a client machine such that the client machine executes the method, or a portion of the method, based on the instructions provided by the diagnostic manager 500. In some embodiments, diagnostic manager 500 can be a CDSS, a CADe, a CADx, a CADq, and/or a CAST system.

The diagnostic manager 500 includes a memory 525, storage 530, an interconnect (e.g., BUS) 520, one or more CPUs 505 (also referred to as processors 505 herein), an I/O device interface 510, I/O devices 512, and a network interface 515.

Each CPU 505 retrieves and executes programming instructions stored in the memory 525 or storage 530. The interconnect 520 is used to move data, such as programming instructions, between the CPUs 505, I/O device interface 510, storage 530, network interface 515, and memory 525. The interconnect 520 can be implemented using one or more busses. The CPUs 505 can be a single CPU, multiple CPUs, or a single CPU having multiple processing cores in various embodiments. In some embodiments, a CPU 505 can be a digital signal processor (DSP). In some embodiments, CPU 505 includes one or more 3D integrated circuits (3DICs) (e.g., 3D wafer-level packaging (3DWLP), 3D interposer based integration, 3D stacked ICs (3D-SICs), monolithic 3D ICs, 3D heterogeneous integration, 3D system in package (3DSiP), and/or package on package (PoP) CPU configurations). Memory 525 is generally included to be representative of a random access memory (e.g., static random access memory (SRAM), dynamic random access memory (DRAM), or Flash). The storage 530 is generally included to be representative of a non-volatile memory, such as a hard disk drive, solid state device (SSD), removable memory cards, optical storage, or flash memory devices. In an alternative embodiment, the storage 530 can be replaced by storage area-network (SAN) devices, the cloud, or other devices connected to the diagnostic manager 500 via the I/O device interface 510 or a network 550 via the network interface 515.

In some embodiments, the memory 525 stores instructions 560 and the storage 530 stores health history 532, diagnostic measures 534, and medical condition probabilities 542. However, in various embodiments, the instructions 560, health history 532, diagnostic measures 534, and medical condition probabilities 542 are stored partially in memory 525 and partially in storage 530, or they are stored entirely in memory 525 or entirely in storage 530, or they are accessed over a network 550 via the network interface 515.

Instructions 560 can be processor-executable instructions for performing any portion of, or all of, any of the methods 100, 200, 300, and/or 400 of FIGS. 1-4. Diagnostic measures 534 can include values 536 (e.g., as discussed in FIG. 3), costs 538 (e.g., as discussed in FIG. 4), and scores 540 that can collectively be used to provide evidence-based decision making in selecting appropriate diagnostic measures to efficiently diagnose medical conditions. Medical condition probabilities 542 can comprise various probabilities for various medical conditions based on health history information of the patient and predicted results of, or actual results of, one or more recommended diagnostic measures. In some embodiments, medical condition probabilities 542 are stored in a vector format with each element of the vector corresponding to a medical condition. In some embodiments, each element comprises an integer data structure (e.g., an int) or a numeric structure with a predefined decimal precision (e.g., a float).

In various embodiments, the I/O devices 512 include an interface capable of presenting information and receiving input. For example, I/O devices 512 can present information to a user interacting with diagnostic manager 500 and receive input from the user.

Diagnostic manager 500 is connected to the network 550 via the network interface 515. Network 550 can comprise a physical, wireless, cellular, or different network.

Embodiments of the present invention can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions can be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or subset of instructions, which comprises one or more executable instructions for implementing the specified logical function(s). In some alternative implementations, the functions noted in the blocks can occur out of the order noted in the Figures. For example, two blocks shown in succession can, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While it is understood that the process software (e.g., any of the instructions stored in instructions 560 of FIG. 5 and/or any software configured to perform any subset of the methods described with respect to FIG. 1-4) can be deployed by manually loading it directly in the client, server, and proxy computers via loading a storage medium such as a CD, DVD, etc., the process software can also be automatically or semi-automatically deployed into a computer system by sending the process software to a central server or a group of central servers. The process software is then downloaded into the client computers that will execute the process software. Alternatively, the process software is sent directly to the client system via e-mail. The process software is then either detached to a directory or loaded into a directory by executing a set of program instructions that detaches the process software into a directory. Another alternative is to send the process software directly to a directory on the client computer hard drive. When there are proxy servers, the process will select the proxy server code, determine on which computers to place the proxy servers' code, transmit the proxy server code, and then install the proxy server code on the proxy computer. The process software will be transmitted to the proxy server, and then it will be stored on the proxy server.

Embodiments of the present invention can also be delivered as part of a service engagement with a client corporation, nonprofit organization, government entity, internal organizational structure, or the like. These embodiments can include configuring a computer system to perform, and deploying software, hardware, and web services that implement, some or all of the methods described herein. These embodiments can also include analyzing the client's operations, creating recommendations responsive to the analysis, building systems that implement subsets of the recommendations, integrating the systems into existing processes and infrastructure, metering use of the systems, allocating expenses to users of the systems, and billing, invoicing (e.g., generating an invoice), or otherwise receiving payment for use of the systems.

What is claimed is:

1. A computer-implemented method comprising:
receiving, by a clinical decision support system (CDSS) executed on a computer, a health history of a patient, the health history comprising a plurality of known diagnostic measures;
generating, by the CDSS, a first number of a first plurality of probabilities corresponding to a plurality of medical conditions based on the health history of the patient;
generating, by the CDSS, a first list of potential diagnostic measures by removing the plurality of known diagnostic measures from a second list of all diagnostic measures;
calculating, by the CDSS, respective values for respective diagnostic measures in the first list of potential diagnostic measures;
calculating, by the CDSS, respective costs for respective diagnostic measures in the first list of potential diagnostic measures;
calculating, by the CDSS, respective scores for respective diagnostic measures in the first list potential of diagnostic measures, wherein respective scores are based on respective values and respective costs; and
outputting, by the CDSS and to a display of the computer, a recommended diagnostic measure having a highest score.

2. The method of claim 1, wherein the respective values are based on predicted changes in the first plurality of probabilities resulting from respective diagnostic measures.

3. The method of claim 1, wherein the respective costs are based on respective availabilities, respective financial costs, and respective side-effects of respective diagnostic measures.

4. The method of claim 1, wherein the respective scores are based on a ratio of a value to a corresponding cost for respective diagnostic measures.

5. The method of claim 1, wherein the health history of the patient comprises medical diagnoses, medical test results, medical procedures, demographic information, social information, and personal information.

6. The method of claim 1, wherein the first number of the first plurality of probabilities is output as a first vector, wherein each element of the first vector corresponds to a respective probability of a respective medical condition.

7. The method of claim 6, wherein calculating respective values further comprises:
selecting a first answer to a first diagnostic measure;
calculating a second plurality of updated probabilities based on the first answer and the first plurality of probabilities;
calculating a first difference between the first plurality of probabilities and the second plurality of updated probabilities, wherein the first difference comprises a largest difference of any corresponding pair of probabilities between the first plurality of probabilities and the second plurality of updated probabilities; and
associating the first difference as a first value of the first diagnostic measure.

8. The method of claim 6, wherein calculating respective values further comprises:
selecting a first answer to a first diagnostic measure;
calculating a second vector comprising a second plurality of updated probabilities based on the first answer and the first plurality of probabilities;
calculating a first difference vector between the first vector and the second vector;
calculating a magnitude of the difference vector; and
associating the magnitude of the difference vector as a first value of the first diagnostic measure.

9. The method of claim 6, wherein calculating respective values further comprises:
selecting a first answer to a first diagnostic measure;
determining a first size of the first vector storing the plurality of probabilities, wherein the first size comprises a quantity of non-zero elements of the first vector;
determining a second size of a second vector storing a second plurality of updated probabilities based on the first answer and the first plurality of probabilities, wherein the second size comprises a quantity of non-zero elements of the second vector;
determining a first difference between the first size and the second size; and
associating the first difference as a first value of the first diagnostic measure.

10. The method of claim 1, wherein calculating respective costs further comprises:
determining, for a first diagnostic measure, a first availability of the first diagnostic measure;
determining, for the first diagnostic measure, a first financial cost associated with the first diagnostic measure;
determining, for the first diagnostic measure, a first side-effect associated with the first diagnostic measure; and
calculating a first cost for the first diagnostic measure using the first availability, the first financial cost, and the first side-effect.

11. A system comprising:
a computer-readable storage medium storing program instructions for a Clinical Decision Support System (CDSS); and
a processor configured to execute the CDSS to perform a method comprising:
receiving, by the CDSS, a health history of a patient, the health history comprising a plurality of known diagnostic measures;
generating, by the CDSS, a first number of a first plurality of probabilities corresponding to a plurality of medical conditions based on the health history of the patient;
generating, by the CDSS, a first list of potential diagnostic measures by removing the plurality of known diagnostic measures from a second list of all diagnostic measures;
calculating, by the CDSS, respective values for respective diagnostic measures in the first list of potential diagnostic measures;
calculating, by the CDSS, respective costs for respective diagnostic measures in the first list of potential diagnostic measures;
calculating, by the CDSS, respective scores for respective diagnostic measures in the first list potential of diagnostic measures, wherein respective scores are based on respective values and respective costs; and
outputting, by the CDSS and to a display of a computer, a recommended diagnostic measure having a highest score.

12. The system of claim 11, wherein the respective values are based on predicted changes in the first plurality of probabilities resulting from respective diagnostic measures.

13. The system of claim 11, wherein the respective costs are based on respective availabilities, respective financial costs, and respective side-effects of respective diagnostic measures.

14. The system of claim 11, wherein the respective scores are based on a ratio of a value to a corresponding cost for respective diagnostic measures.

15. The system of claim 11, wherein the first number of the first plurality of probabilities is output as a first vector, wherein each element of the first vector corresponds to a respective probability of a respective medical condition.

16. The system of claim 15, wherein calculating respective values further comprises:
selecting a first answer to a first diagnostic measure;
calculating a second plurality of updated probabilities based on the first answer and the first plurality of probabilities;
calculating a first difference between the first plurality of probabilities and the second plurality of updated probabilities, wherein the first difference comprises a largest difference of any corresponding pair of probabilities between the first plurality of probabilities and the second plurality of updated probabilities; and
associating the first difference as a first value of the first diagnostic measure.

17. The system of claim 15, wherein calculating respective values further comprises:
selecting a first answer to a first diagnostic measure;
calculating a second vector comprising a second plurality of updated probabilities based on the first answer and the first plurality of probabilities;
calculating a first difference vector between the first vector and the second vector;
calculating a magnitude of the difference vector; and
associating the magnitude of the difference vector as a first value of the first diagnostic measure.

18. The system of claim 15, wherein calculating respective values further comprises:
selecting a first answer to a first diagnostic measure;
determining a first size of the first vector storing the plurality of probabilities, wherein the first size comprises a quantity of non-zero elements of the first vector;
determining a second size of a second vector storing a second plurality of updated probabilities based on the first answer and the first plurality of probabilities, wherein the second size comprises a quantity of non-zero elements of the second vector;
determining a first difference between the first size and the second size; and
associating the first difference as a first value of the first diagnostic measure.

19. The system of claim 11, wherein calculating respective costs further comprises:
determining, for a first diagnostic measure, a first availability of the first diagnostic measure;
determining, for the first diagnostic measure, a first financial cost associated with the first diagnostic measure;
determining, for the first diagnostic measure, a first side-effect associated with the first diagnostic measure; and
calculating a first cost for the first diagnostic measure using the first availability, the first financial cost, and the first side-effect.

20. A computer program product comprising a computer-readable storage medium having program instructions embodied therewith, wherein the computer-readable storage medium is not a transitory signal per se, the program instructions executable by a processor to cause the processor to perform a method comprising:
receiving, by a clinical decision support system (CDSS) executed on a computer, a health history of a patient, the health history comprising a plurality of known diagnostic measures;
generating, by the CDSS, a first number of a first plurality of probabilities corresponding to a plurality of medical conditions based on the health history of the patient;
generating, by the CDSS, a first list of potential diagnostic measures by removing the plurality of known diagnostic measures from a second list of all diagnostic measures;
calculating, by the CDSS, respective values for respective diagnostic measures in the first list of potential diagnostic measures;
calculating, by the CDSS, respective costs for respective diagnostic measures in the first list of potential diagnostic measures;
calculating, by the CDSS, respective scores for respective diagnostic measures in the first list potential of diagnostic measures, wherein respective scores are based on respective values and respective costs; and
outputting, by the CDSS and to a display of the computer, a recommended diagnostic measure having a highest score.
